(19) United States
(12) Patent Application Publication
(10) Pub. No.: US 2014/0154184 A1
(43) Pub. Date: Jun. 5, 2014

(54) TIME-GATED FLUORESCENCE IMAGING WITH SI-CONTAINING PARTICLES

(71) Applicants: Michael J. Sailor, La Jolla, CA (US); Luo Gao, La Jolla, CA (US); David J. Hall, San Diego, CA (US); Robert Mattrey, San Diego, CA (US)

(72) Inventors: Michael J. Sailor, La Jolla, CA (US); Luo Gao, La Jolla, CA (US); David J. Hall, San Diego, CA (US); Robert Mattrey, San Diego, CA (US)

(73) Assignee: THE REGENTS OF THE UNIVERSITY OF CALIFORNIA, Chicago, IL (US)

(21) Appl. No.: 14/113,960

(22) PCT Filed: Apr. 27, 2012
(86) PCT No.: PCT/US2012/035447

§ 371 Date: Feb. 10, 2014

Related U.S. Application Data
(60) Provisional application No. 61/480,149, filed on Apr. 28, 2011.

(51) Int. Cl. A61K 49/00

(52) U.S. Cl. CPC A61K 49/0021
USPC 424/9.6

(57) ABSTRACT

A method for imaging leverages the fluorescence lifetime of a fluorescent Si-containing particle to distinguish from background fluorescence. A particle is introduced into tissue. An excitation light pulse is applied to excite luminescence from the fluorescent Si-containing particle. Time-gated measuring of a responsive luminescence signal identifies the particle. In preferred embodiments the particle is coated or encapsulated with an organic material. The fluorescence lifetime of particles can be controlled during manufacture, such as by oxidation levels, quenching treatments, or by aging. This permits introducing and using groups of particles in imaging that have unique lifetimes and multiple time gating can be used to identify different particles or to monitor the change in lifetime of a single set of particles as they respond to a biochemical stimulus. The particles can also be functionalized for affinity to particular tissues and can be loaded with treatment molecules.

### PRIORITY CLAIM AND REFERENCE TO RELATED APPLICATION

The application claims priority under 35 U.S.C. §119 from prior provisional application Ser. No. 61/480,149, which was filed on Apr. 28, 2011 and is incorporated by reference herein. This application is also a continuation-in-part of prior pending U.S. application Ser. No. 13/202,271 and claims priority under 35 U.S.C. §120 from that application, which is incorporated by reference herein.

### STATEMENT OF GOVERNMENT INTEREST

This invention was made with government support under Contract Nos. CA119335 and CA124427 awarded by the National Institutes of Health. The Government has certain rights in this invention.

### FIELD

A field of the invention is imaging/diagnostic agents and methods. Another field of the invention is luminescent nanostructures.

### BACKGROUND

Fluorescence imaging is one of the most versatile and widely used visualization methods in biomedical research because of its high sensitivity, high spatial resolution, low cost, and non-invasive nature. In this method, a fluorescent probe molecule or nanoparticle is used to enhance contrast of the image in desired regions or to identify specific features, molecules, or tissues. However, in vivo fluorescence imaging has not been widely applied in clinical practice due to the lack of specific imaging agents, shallow tissue penetration of the exciting or emitting wavelengths, and ubiquitous background tissue autofluorescence.

Common fluorescence imaging or detection techniques normally use wavelength (energy or color) based methods to discriminate the fluorescent probe from background or from other probes. Any fluorescent species emits light with a characteristic lifetime (time required for the intensity of fluorescence to decay to 1/e after a short excitation pulse) that is determined by the rates of radiative and nonradiative decay processes available to the fluorescent probe. Conventional fluorescent probes based on organic molecules or quantum dots normally display short fluorescence lifetimes (on the order of a few nanoseconds). These lifetimes are comparable to the lifetimes of naturally occurring species in tissues and cellular media that are responsible for autofluorescence. This makes it difficult to separate the fluorescence signal of the probe from background fluorescence in the time domain. There is also inevitably some leakage of the excitation light through the emission filter. As such, an intensity image contains fluorescence from the fluorescent probe, tissue autofluorescence, and excitation light leakage.

In vivo fluorescence imaging has become a dominant preclinical molecular imaging modality. Several commercial instruments can readily provide in vivo fluorescence intensity images of fluorescent probes in small animals. However, tissue autofluorescence continues to be a nemesis which can confound interpretation of fluorescence intensity images. Recently, spectral-unmixing methods have been applied to correct for tissue autofluorescence.

To provide the desired image of just the fluorescent probe requires discrimination of these intensity sources with further information. One approach is to measure intensity as a function of emission wavelength, i.e. fluorescence spectroscopy, as different fluorophores have different emission spectra which can be discriminated from each other and the excitation light source. Since these spectra overlap, the measured mixed spectral signal must be decomposed into the fluorescent probe contribution with spectral-unmixing methods. An alternative method is to measure the intensity decay as a function of time (nanosecond timescale) following an excitation light pulse as different fluorophores have different fluorescence lifetimes which can be discriminated from each other and the excitation light pulse. Here, the measured decay curve must be decomposed into the fluorescent probe contribution with lifetime-unmixing methods.

Since the 1980s there has been interest in imaging tissues, cells, and whole organisms using the time-dependence of fluorescence (“fluorescence lifetime”) instead of the wavelength dependence, but this goal has proved elusive due to the interference fluorescence signals discussed above. Exogenous dyes are favored for imaging, but the fluorescence emanating from molecules that naturally occur in biological systems generates an interference when operators attempt to image the exogenous dyes that they have added to the tissues to provide them with a useful diagnostic measurement (of cell viability or metabolic activities, for instance). Tissue autofluorescence often shows up in the same wavelength range as the exogenous probe, and so it limits the sensitivity of the assay. The molecules used in all commercial exogenous dyes and those natively present all tend to have very short excited state lifetimes—that is, they continue to emit light for a very short time after the excitation source is turned off. Lifetimes of 10 nanoseconds or less are typical.

Time-gated bio imaging has been considered previously with exogenous dyes and other probes. See, e.g., Cubeddu et al., “Time-Gated Fluorescence Imaging for Diagnosis of Tumors in a Murine Model,” Photochemistry and Photobiology 57 480-485 (1993). The lifetime of the exogenous dyes is close to the lifetime of tissue autofluorescence, and research on these techniques is not believed to have continued. Instead, researchers have favored wavelength-resolved methods over time-resolved methods. Although molecules and materials with longer fluorescence lifetimes have been proposed for time-gated imaging, these have limited utility due to their cytotoxicity or systemic toxicities. See, e.g., Fisher, B., et al., “Room-Temperature Ordered Photon Emission from Multiexciton States in Single CdSe Core-Shell Nanocrystals,” Physical Review Letters 94 (2005); Dalian, M. et al, “Time-Gated Biological Imaging by use of Colloidal Quantum Dots,” Optics Letters 26 (11) 825-827 (2001).

### SUMMARY OF THE INVENTION

An embodiment of the invention is a method for imaging. In the method, a fluorescent Si-containing particle is introduced into tissue. An excitation light pulse is applied to excite luminescence from the fluorescent Si-containing particle. Time-gated measuring of a responsive luminescence signal identifies the particle. In preferred embodiments the particle is coated or encapsulated with an organic material. The fluorescence lifetime of particles can be controlled during manufacture, such as by oxidation levels, quenching treatments, or by aging. This permits introducing and using groups of particles in imaging that have unique lifetimes and multiple time gating can be used to identify different particles. The particles can also be functionalized for affinity to particular tissues and can be loaded with treatment molecules.

### BRIEF DESCRIPTION OF THE DRAWINGS

FIG. 1 is a schematic diagram of the synthesis of luminescent porous silicon nanoparticles that was used to produce particles for experiments demonstrating late-gate imaging methods of the invention;

FIG. 2 illustrates a lifetime decay curve for experimentally produced porous silicon nanoparticles;

FIGS. 3A-3C show in vivo fluorescence imaging and data of porous silicon nanoparticles administered at a mouse foot pad.

### DESCRIPTION OF PREFERRED EMBODIMENTS

Preferred embodiment methods of the invention provide imaging of tissue with a fluorescent silicon containing particle that has a fluorescence lifetime that is longer than the autofluorescence lifetime of the subject tissue. The fluorescence lifetime of the particle is preferably substantially longer than the autofluorescence lifetime of the tissue, meaning that the particle provides readily measurable fluorescence after the autofluorescence of the tissue has diminished to negligible level. Fluorescence response is measured with late time-gating after the light pulse has ceased to avoid tissue autofluorescence, i.e., the measurement is made after the time when the tissue autofluorescence has diminished to a negligible level. This encompasses only sensing the signal after the late time-gate period or sensing the signal continually but only analyzing the signal after the contribution from autofluorescence has diminished to a low level.

Porous and other silicon nanoparticles can possess very long-lived excited states (up to a few hundred microseconds and a millisecond). This permits complete elimination of tissue autofluorescence that is not existent after few tens of nanoseconds post excitation. Autofluorescence typically decays to a negligible level after about 1-10 nanoseconds based upon the surrounding tissues and is typically completely eliminated after about 20 to 30 nanoseconds, so the time-gated data reveal only the porous silicon nanoparticles and no background. The type of tissue determines the specific amount of autofluorescence decay. Endogenous fluorophores responsible for tissue autofluorescence have decay lifetimes of ˜1-10 ns, depending on the type of tissue. See, e.g., Berezin, M. Y. et al., “Fluorescence Lifetime Measurements and Biological Imaging,” Chemical Reviews 110, 2641-2684 (2010).

Long lifetime fluorescent particles therefore provide the fluorescence signal. The signal does not require post processing to eliminate background autofluorescence. The late time-gating (either via the sensor or software analysis of the sensor's signal) also avoids background signals associated with leakage of the excitation light through the emission filter as the excitation light pulse has completed before the late time-gate sensing occurs. The methods of the invention can use intensity images and require no wavelength analysis, as an acquired late-gate intensity signal will have an intensity image that contains fluorescence from the fluorescent particle, but lacks any non-negligible signal from tissue autofluorescence or excitation light leakage. Methods of the invention have clear potential for clinical application because silicon nanoparticles are non-toxic and have been demonstrated to degrade into renally cleared components in a relatively short period of time with little or no evidence of systemic or cellular toxicity.

Silicon occurs in nature and is widely distributed as various forms of the compounds silicon dioxide (silica) or silicates. Silicon is used in the electronics industry where substantially pure and highly pure silicon are used for the formation of wafers. Pure silicon is used to produce ultra-pure silicon wafers used in the semiconductor industry, in electronics and in photovoltaic applications. Ultra-pure silicon can be doped with other elements to adjust its electrical response by controlling the number and charge (positive or negative) of current carriers. Such control is desirable for transistors, solar cells, integrated circuits, microprocessors, semiconductor detectors and other semiconductor devices which are used in electronics and other high-tech applications. Silicon is an essential trace element that is linked to the health of bone and connective tissues.

Silicon dioxide refers to the compound SiO₂ (sometime referred to as silica). Silicon dioxide is formed when silicon is exposed to oxygen (or air or water). A thin layer (approximately 1 nm or 10 Å) of so-called “native oxide” is formed on the surface when silicon is exposed to air under ambient conditions. Higher temperatures and alternate environments (such as aqueous phase) are used to grow layers of silicon dioxide on silicon. Silicon dioxide is inert and harmless.

Preferred embodiments of the invention provide a method of using a fluorescent (luminescent) porous silicon-based nanoparticle that has a long lifetime compared to most organic molecules and to background tissue autofluorescence to conduct high fidelity imaging based on time-gated fluorescence imaging. The invention provides porous silicon containing nanostructures that are luminescent and/or modified with a quenching/stabilizing component. In a preferred embodiment of the invention, the porous silicon containing nanostructure has high oxide content, which can be achieved by a long water oxidation time of a month or up to about 3 months. The invention provides a porous nanoparticle prepared from elemental silicon with a long luminescence lifetime and potentially containing a plurality of quenching/stabilizing molecules that can adjust the lifetime and intensity of luminescence from the nanoparticle. A preferred embodiment stabilizing molecule includes Polyethylene glycol (PEG)-silane. Other silicon nanoparticles are also known to fluoresce and can be treated to affect fluorescence lifetime. Such silicon nanoparticles can also be used with the invention. See, e.g., Peng Shen et al., “Stable and Color-Tunable Fluorescence from Silicon Nanoparticles Formed by Single-Step Plasma Assisted Decomposition of SiBr4,” J. Mater. Chem., 20, 1669-1675 (2010); Mader et al, “Fluorescent Silica Nanoparticles,” Ann N Y Acad Sci. 1130:218-23 (2008); Nayfeh et al, “Effect of Ultrathin Oxides on Luminescent Silicon. Nanocrystallites,” Appl. Phys. Lett. 73, 841 (1998).

The quenching component can be a species that can accept energy or charge from the excited state(s) in the fluorescent silicon nanoparticle, such as a porphyrin, a polycyclic aromatic hydrocarbon, a redox-active molecule, or a metal in its metallic or ionic state. These species can be expected to introduce a non-radiative excitation decay channel into the photosystem, decreasing the observed emission lifetime. See, Sailor, M. et al. “Photoluminescence-Based. Sensing with Porous Silicon Films, Microparticles, and Nanoparticles,” Adv. Func. Mater. 2009, 19 (20), 3195-3208. The quenching component can be physically or chemically adsorbed to the fluorescent silicon nanoparticle in order to place it in proximity sufficient to allow the energy or charge transfer process to take place. Biological or chemical processes that solubilize or degrade these quenching components are expected to then remove the quenching pathway, resulting in an increase in the emission lifetime of the nanoparticle probe. This change in emission lifetime can then be used to indicate the biological state of the tissue being probed. Although these quenching chemistries have been demonstrated previously, the harnessing of the chemistries to accomplish lifetime imaging, and in particular using said lifetime imaging as a probe of biological state is not believed to have been proposed or reported previously. The fluorescent nanoparticle can also contain molecules to improve the biocompatibility of the material, such as poly(ethylene glycol), dextran, aminated dextran, or chitosan. The fluorescent nanoparticle can also contain or carry molecules to improve the ability of the material to target specific tissues, such as the pentapeptide CREKA, the tripeptide ROD, the cyclic peptide iRGD (Sugahara, K. N. et al, Coadministration of a Tumor-Penetrating Peptide Enhances the Efficacy of Cancer Drugs. Science 2011, 328 (5981) 1031-1035), an antibody, or an aptamer. Imaging of the nanoparticles with time-gated detection (either via sensing after a predetermined period of time after the excitation light source has been terminated or continually sensing by monitoring the phase shift of light emission relative to the phase of an intensity oscillating excitation source) enables higher fidelity imaging by eliminating background signals from tissue-based autofluorescence, other short-lived fluorescent interferents and any short-lived excitation-light filter-leakage signals.

Preferred embodiment methods use porous silicon nanoparticles (pSiNP), for example, that can emit visible to near-IR light under photoexcitation and have luminescence lifetimes >10 nanoseconds. A preferred embodiment of the invention is a time-gated fluorescence imaging method that uses a biodegradable porous Si-containing nanostructure comprising a luminescence emission maximum that appears in the wavelength range of about 400 nm to about 1000 nm and a luminescence lifetime between about 10 nanosecond to 1 millisecond. In preferred embodiments, late-gating provides a signal after 100 nanoseconds. The autofluorescence of most tissue typically becomes negligible after about 10 nanoseconds with a typical excitation pulse in the range of ˜300 to 1000 nm, and should for all cases and tissues be negligible after 1.5 nanoseconds. Accordingly, the fluorescence lifetime of the particle extends well beyond the autofluorescence range and permits late-gated measurement. The silicon material can be a silicon dioxide material or can be both elemental silicon and a silicon dioxide material. Other embodiments use solid fluorescent silicon nanoparticles, which are known in the art to produce controllable fluorescence and be suitable for introduction into tissues. Particle sizes of solid and porous particles can be in the range of 1 nanometer and 100,000 nanometers. Biodegradable silicon nanostructures of the invention can be characterized as non-toxic.

The silicon particles used in imaging methods of the invention also have other advantages. In contrast to many nanomaterials (e.g., carbon nanotubes (CNT), gold nanoparticles (GN), and quantum dots (QD)), used in high fidelity biological imaging, particles of the invention are biodegradable, i.e., the particles degrade into renally cleared components in a relatively short period of time with little or no evidence of systemic or cellular toxicity. Preferred porous silicon or porous silicon oxide nanoparticles also provide a porous nanostructure with the capacity to bind a plurality of the same or different molecules or complexes that can modulate the luminescence lifetime of the nanoparticles, and which can be engineered to modulate the fluorescence lifetime in response to specific stimuli. A biodegradable porous nanostructure of the invention can be coated or encapsulated within an organic or polymeric material containing quenching species that can change the fluorescence lifetime of the porous nanostructure. Suitable exemplary quenching species include porphyrins, polycyclic aromatic hydrocarbons, redox-active molecules, or metals in their metallic or ionic states. Additional exemplary quenching species include aromatic compounds, heterocyclic compounds, or metal-containing coordination compounds or combinations of these compounds. Quenching is useful if the sensor is used to measure response of biological/chemical triggers. See, Sailor et al., “Photoluminescence Quenching and the Photochemical Oxidation of Porous Silicon by Molecular Oxygen,” LANGMUIR Vol. 13, issue 17 pp. 4652-4658 DOI: 10.1021/la960535z (1997). Suitable exemplary organic or polymeric materials include enzyme-cleavable polypeptide linkers, oligonucleotides, dextran, aminated dextran, chitosan, polylactic acid, polyglycolic acid, collagen, fibrin, copolymers of polylactic acid and polyglycolic acid, and co-polymers of dextran and polylactic acid or combinations of these materials. The organic or polymeric materials can be considered to be bio-responsive linkers, wherein the action of endogenous biological species (such as proteolytic enzymes, matrix metalloproteinases, nucleases, or collagenases) on the organic or polymeric materials will result in release of the quenching species from the effective vicinity of the nanoparticle and thus provide an increase in the emission lifetime of the nanoparticle. This increase in emission lifetime of the nanoparticle can then be used as an indication of the physiological state (up- or down-regulation of specific genes, presence or concentration of said biological species) of the tissues being probed with the time-gated imaging method.

A preferred organic is one of or a derivative of one of benzene, toluene, tetrahydrofuran, xylene, diethyl ether, formic acid, benzophenone, naphthalene, pyrene, anthracene, 9,10-dimethylanthracene, ethanol, porphyrin, dodecyltrimethyl ammonium bromide, sodium dodecylsulfonate, fluorescein, ruthenium tris-bipyridine or ferrocene. A preferred inorganic material contains one of gold, silver, copper, platinum, iron, cobalt, chromium, manganese, zinc, ruthenium, cadmium selenide, zinc sulfide, elemental carbon, or carbon nanotubes.

Preferred particles for use with imaging method of the invention can be prepared as follows. A silicon wafer is etched to generate a porous nanostructured film. The porous nanostructured film is separated from the silicon wafer substrate and then fractured to generate nanoparticles of sizes between 1 nanometers and 100,000 nanometers. Preferred embodiments include a crystalline silicon feature within the porous nanoparticle, responsible for light emission, of size <10 nm. In other preferred embodiments, the active emissive center consists of an interfacial silicon/silicon oxide material of size <500 nm. A preferred porous silicon nanostructure has pores of diameters between 1 and 200 nm. In a preferred embodiment, an organic or polymeric coating increases the circulatory time of the biodegradable porous nanostructure in vivo.

Etching conditions and materials permit the porosity and pore size to be controlled. For example, the control provided by doping can be used to determine the porosity and pore size of porous silicon made by electrochemical etch. Methods for controlling the pore size and distribution are disclosed, for example, in the following patent publications of some of the present inventors and colleagues: Sailor et al. U.S. Pat. No. 5,338,415; Sailor et al. U.S. Pat. No. 7,433,811; Sailor et al. U.S. Pat. No. 7,903,239; Sailor U.S. Pat. No. 7,042,570; Sailor et al. U.S. Pat. No. 7,942,989; Sailor et al. U.S. Published Application No. 2007/0051815; & Sailor et al. U.S. Published Application No. 2005/0042764.

The nanoparticles are activated in an aqueous solution, which can be, for example, pure water, sodium hydroxide, hydrogen peroxide or an aqueous solution containing the borate ion. The particles can also be loaded with a molecule or agent that changes their luminescence lifetime: porphyrins, polycyclic aromatic hydrocarbons, redox-active molecules, metals in their metallic or ionic states, aromatic compounds, heterocyclic compounds, metal-containing coordination compounds, silicon carbide, silicon nitride, or combinations of these compounds. These formulations provide the ability to differentiate particles based on differences in lifetime. A biocompatible agent can be adsorbed to the nanostructure to increase the circulation half-life or circulatory time in vivo. A tissue-specific targeting agent can also be loaded onto the particle or its coating. A therapeutic agent can also be loaded into the pores of the nanoparticles. Nanoparticles of the invention loaded with a therapeutic agent provide a pharmaceutical composition having pharmaceutically acceptable carrier that is a biodegradable porous nanostructure.

Preferred methods of the invention include filtering the fractured porous nanoparticles after fracturing; and activating the size fractionated nanoporous material by incubating the material in an aqueous solution to obtain a luminescent porous silicon nanoparticle. Details about fabrication of porous silicon and silicon dioxide for carrying drugs and other molecules can be found in Sailor et al. U.S. Published Patent Application Serial 201110300222

Additionally, due to their porous nature, the nanoparticles can carry additional species such as drugs, smaller nanoparticles, fluorescence sensitizers or fluorescence quenching agents. Details about preparations of porous silicon and silicon dioxide for carrying drugs and other molecules can be found in Sailor et al. U.S. Published Patent Application Serial 201110300222.

For a predetermined late time-gate of sensing or analyzing a fluorescence signal, e.g. 20 nanoseconds or more after the excitation pulse, and preferably after 100 nanoseconds, only the fluorescence intensity from the nanoparticle persists where the excitation pulse and tissue autofluorescence have decayed to negligible levels. As such, the desired image of just the fluorescent nanoparticle(s) can be directly generated from the late time-gated data. This simple and straightforward approach avoids any unmixing methods since there is no temporal overlap of the desired signal and background and autofluorescence.

The present inventors have also determined that the fluorescence lifetime of particles changes with surface treatments or oxidations, and also changes with aging (either in vitro or in vivo), which provides the ability to differentiate particles based on differences in lifetime. In a preferred imaging method, two or more particle types (e.g., porous Si nanoparticles containing different surface chemistries that would allow them to target different tissues, or porous Si nanoparticles with different emission lifetimes due to their possession of different quencher molecules, with different bio-responsive linkers) are introduced into tissue(s). Fluorescence decay at different rates permits discriminating between the different particle types. Particles aged for different times ex vivo, or particles injected in vivo at different times can also achieve the same effect. With the invention, therefore, just as the spectral wavelength of emission of different dyes can be distinguished based on their color in the spectral domain, the porous Si nanoparticles with different lifetimes can be distinguished in the time domain. The invention thus provides for time-domain imaging with porous Si nanoparticles either in vitro or in vivo that accomplishes tasks achieved in the spectral domain with more complex processing.

The porous silicon nanoparticles of the invention provide a device and method for time-gated fluorescence imaging and tissue (e.g., tumor, brain, cardiac, skin) monitoring. For example, the method of time gating the emission of the nanoparticles to eliminate the fast decaying background signal (such as tissue autofluorescence, spectral bleed through, etc.) and only collect the long-lived emission from the silicon nanoparticles can significantly enhance the sensitivity and signal to noise ratio of the imaging method.

A preferred embodiment biodegradable porous silicon containing particle of the invention can emit visible to near-IR light under photoexcitation and that has long luminescence lifetime compared to background (tissue) autofluorescence or excitation spectral bleed through, which allows the method of using time gating of the emission of the nanoparticles to eliminate the fast decaying background signal and only collect the long-lived emission from the device. The nanomaterials can have controllable rates of self-destruction, through which components could be hierarchically degraded into harmless, renally-cleared products after performing their in vivo function. Methods, nanostructures and systems of the invention address an important and believed unmet biomedical need for a minimally invasive imaging and diagnostic system and methods of using the system that would enable higher sensitivity and signal to noise ratio of fluorescence imaging and fluorescence assays.

Preferred embodiments of the invention will now be discussed with respect to experiments and data relating to the experiments. The drawings presented with the discussion of experimental results may include schematic representations, which will be understood by artisans in view of the general knowledge in the art and the description that follows. Features may be exaggerated in the drawings for emphasis, and features may not be to scale.

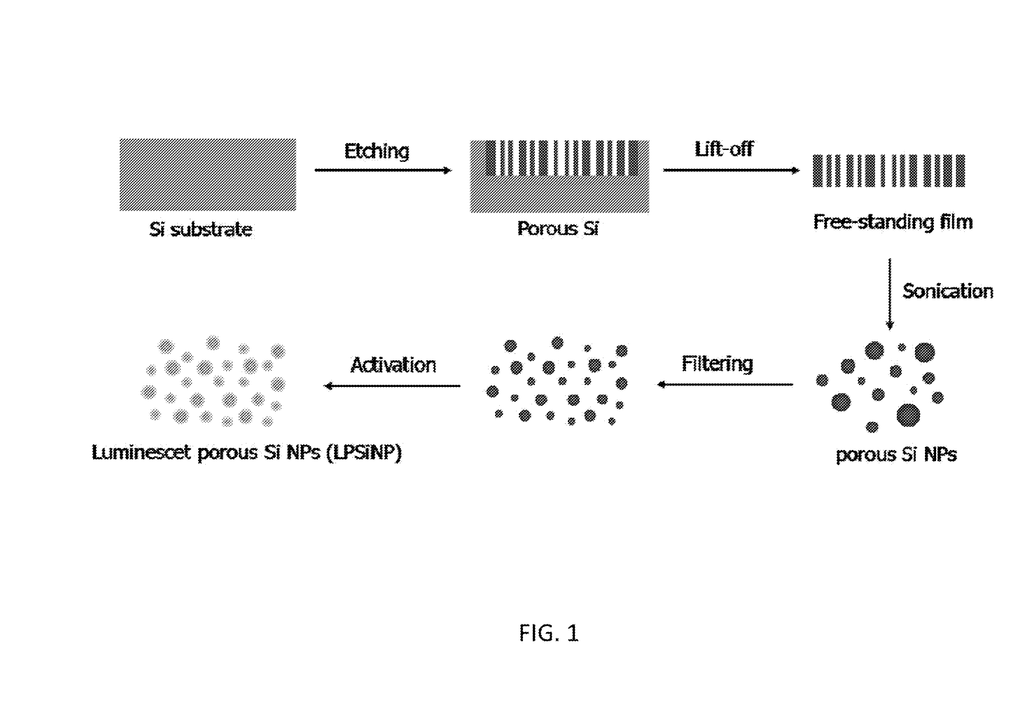

FIG. 1 is a schematic diagram of the synthesis of luminescent porous silicon nanoparticles that was used to produce particles for experiments demonstrating late-gate imaging methods of the invention. A porous silicon layer 10 is etched into a single-crystal silicon substrate 12 in ethanolic HF solution. Pore morphology and pore size can be varied by controlling the current density, the type and concentration of dopant, the crystalline orientation of the wafer, and the electrolyte concentration in order to form macro-, meso-, and micropores. Pore sizes ranging from 1 nm to a few microns can be prepared in this process. The type of dopant in the original silicon wafer is important because it determines the availability of valence band holes that are the key oxidizing equivalents in the electrocorrosion reaction. In general the relationships of dopant to morphology can be segregated into four groups based on the type and concentration of the dopant: n-type, p-type, highly doped n-type, and highly doped p-type. By “highly doped,” is meant dopant levels at which the conductivity behavior of the material is more metallic than semiconducting, although the material still retains the fundamental band structure of a semiconductor. For n-type silicon wafers with a relatively moderate doping level, exclusion of valence band holes from the space charge region determines the pore diameter. Quantum confinement effects are thought to limit pore size in moderately p-doped material. For both dopant types the reaction is crystal face selective, with the pores propagating primarily in the <100> crystallographic direction of the single crystal.

The porosity of a growing porous Si layer is proportional to the current density being applied, and it typically ranges between 40 and 80%. Pores form at the Si/porous Si interface, and once formed, the morphology of the pores does not change significantly for the remainder of the etching process. However, the porosity of a growing layer can be altered by changing the applied current. The film will continue to grow with this new porosity until the current changes.

Stain etching is an alternative to the electrochemical method for fabrication of porous Si powders. The term stain etching refers to the brownish or reddish color of the film of porous Si that is generated on a crystalline silicon material subjected to the process. In the stain etching procedure, a chemical oxidant (typically nitric acid) replaces the power supply used in the electrochemically driven reaction. HF is a required ingredient, and various other additives are used to control the reaction. Stain etching generally is less reproducible than the electrochemical process, although recent advances have improved the reliability of the process substantially. Porous Si powders prepared by stain etch are commercially available.

The entire porous nanostructure 10 is removed from the Si substrate 12. This freestanding hydrogen-terminated porous silicon film 10 is then placed in an aqueous solution and fractured into particles 14 by ultrasonication. The porous layer can be removed from the Si substrate by “electropolishing” or “lift-off.” The etching electrolyte is replaced with one containing a lower concentration of HF and a current pulse is applied for several seconds. The lower concentration of HF results in a diffusion limited situation that removes silicon from the crystalline Si/porous Si interface faster than pores can propagate. The result is an undercutting of the porous layer, releasing it from the Si substrate. The freestanding porous Si film can then be removed with tweezers or a vigorous rinse.

The particles are then filtered through a porous filtration membrane or separated by centrifugation to obtain porous silicon particles with a desired size. Finally, the particles are incubated in an aqueous solution to activate their luminescence, creating luminescent particles 16. Quenching species can be physically or chemically absorbing to the nanoparticles to change luminescence lifetime. As an example, amino-Polyethylene glycol (PEG)-silane was tested and can slightly decrease the lifetime after conjugation with the particles. This provides the ability to have different sets of nanoparticles with different predetermined luminescence lifetimes. Conventional lithography or other patterning methods can also be used to control the fracturing if particles with more uniform shapes are desired.

Oxidation of the porous Si surface in an oxidizing aqueous environment for a sufficient period of time provides a luminescent material. Such luminescent materials are useful for monitoring in vivo conditions or biological changes in cells or environments where toxicity is an issue. Various oxidizing aqueous buffers are known. For example, in one embodiment, a borate aqueous buffer is useful. Borates in chemistry are chemical compounds containing boron oxoanions, with boron in oxidation state +3. The simplest borate ion is the trigonal planar, BO₃³⁻, although many others are known.

Slow oxidation of the porous Si surface by dimethyl sulfoxide (DMSO), when coupled with dissolution of the newly formed oxide by HF, is a mild means to enlarge the pores in porous Si films. Aqueous solutions of bases such as KOH can also be used to enlarge the pores after etching. Electrochemical oxidation, in which a porous Si sample is anodized in the presence of a mineral acid such as H₂SO₄, yields a fairly stable oxide. Oxidation imparts hydrophilicity to the porous structure, enabling the incorporation and adsorption of hydrophilic drugs or biomolecules within the pores. Aqueous oxidation in the presence of various ions including Ca²⁺ generates a calicified form of porous Si that has been shown to be bioactive and is of particular interest for in vivo applications. Calcification can be enhanced by application of a DC electric current.

The mechanical instability of porous Si is directly related to the strain that is induced in the film as it is produced in the electrochemical etching process, and the volume expansion that accompanies thermal oxidation can also introduce strain. Mild chemical oxidants presumably attack porous Si preferentially at Si—Si bonds that are the most strained, and hence most reactive. As an alternative, nitrate is a stronger oxidant, and nitric acid solutions are used extensively in the preparation of porous Si particles from silicon powders by the chemical stain etching method mentioned above.

Smaller pores provide more surface area and expose more sites for attack of aqueous media. The smaller porous filaments within the film yield greater dissolution rates, providing a convenient means to control degradation rates of the porous Si host. A fractionated mixture of particles can be filtered to obtain a desired average particle size. For example, a filter can be used to obtain nanostructures smaller than 220 nm. With its high surface area, porous Si is particularly susceptible to air or water oxidation. Once oxidized, nanophase SiO₂ readily dissolves in aqueous media, and surfactants or nucleophiles accelerate the process. Si—O bonds are easy to prepare on porous Si by oxidation, and a variety of chemical or electrochemical oxidants can be used. Thermal oxidation in air tends to produce a relatively stable oxide, in particular if the reaction is performed at >600° C. Ozone oxidation, usually performed at room temperature, forms a more hydrated oxide that dissolves quickly in aqueous media. Milder chemical oxidants, such as dimethyl sulfoxide, benzoquinone, pyridine, borate, aqueous buffer, or hydrogen peroxide can also be used for this reaction. Mild oxidants are sometimes preferred because they can improve the mechanical stability of highly porous Si films, which are typically quite fragile.

The ability to easily tune the pore sizes and volumes during the electrochemical etch is a unique property of porous Si that is very useful for drug, vaccine, or antigen delivery applications. Other porous materials generally require a more complicated design protocol to control pore size, and even then, the available pore sizes tend to span a limited range. With electrochemically prepared porous Si, control over porosity and pore size is obtained by adjusting the current settings during etching. Typically, larger current density produces larger pores. Large pores are desirable when incorporating sizable molecules or drugs within the pores. Pore size and porosity is important not only for drug loading; it also determines degradation rates of the porous Si host matrix.

In experiments, an electrochemical etch was performed by application of a constant current density of 200 mA/cm² for 150 s in an aqueous HF/ethanol electrolyte. The freestanding film was obtained by application of a current pulse of 4 mA/cm² for 250 s in an aqueous HF/ethanol electrolyte. The freestanding film was then fractured into nanoparticles by exposure to ultrasound. The particles were further isolated by passing a solution of the nanoporous material through a 0.22 to 0.45 μm filtration membrane. The nanoparticles were activated by incubating the nanoparticles in an aqueous borate buffer or hydrogen peroxide. Size fractionated nanoparticles were activated in deionized water for approximately 2 weeks.

Imaging methods of the invention were conducted with particles thus manufactured. Time gating the emission of the porous silicon nanoparticles was demonstrated to effectively eliminate the fast decaying background signal (such as tissue autofluorescence, spectral bleed through, etc.) and use the relatively long-lived light emission from the silicon nanoparticles of the invention. The experiments demonstrated the ability to use the late-gating method to accomplish simple time domain imaging of particles introduced into tissue.

Preliminary in vivo results were obtained with time domain imaging. Specifically, a time domain fluorescence preclinical imager (eXplore Optix™) was used to acquire in vivo data from mice injected with the silicon nanoparticle. Standard fluorescence intensity images were compared with late time-gated images where the latter only shows the silicon nanoparticle and the former shows additional interfering signal relating to the fast decaying background signals (such as tissue autofluorescence, spectral bleed through, etc.).

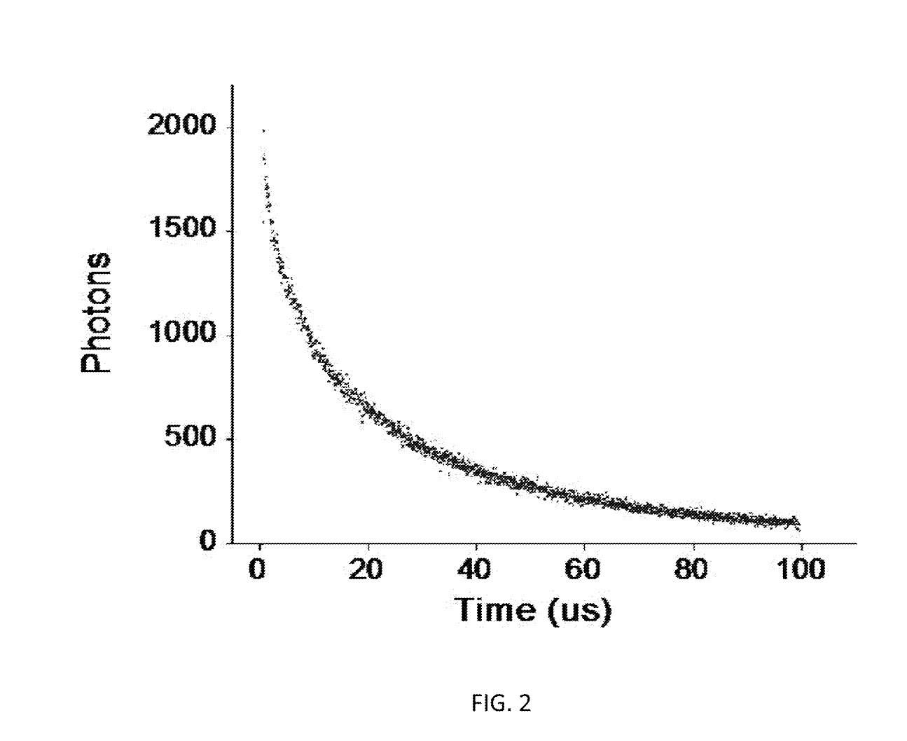

FIG. 2 illustrates a lifetime decay curve for experimentally produced porous silicon nanoparticles. The lifetime is approximately 12 μs (calculated as the luminescence intensity drops to 1/e of the initial intensity). However, significant measurable luminescence remains past 12 μs and can be used for late gate measurement.

In the particular data set in FIG. 2, the period of up to about 40-50 μs provides non-negligible luminescence. This also can be altered with different treatments. Specifically, the luminescence lifetime of porous silicon nanoparticles can be tuned through physical or chemical modification of the material. For example, particle size, pore diameter, oxide layer, or polyethylene glycol (PEG) coating can alter the luminescence lifetime of porous silicon nanoparticles, which can allow to control the lifetime of porous Si-based nanoparticles by incorporation of selective quenchers, and use of the characteristic emissive timescale as an in vivo or in vitro probe of biochemical processes. Table 1 below provides examples of particles that have different luminescence lifetimes calculated according to the 1/e of the initial intensity.

TABLE 1
Fluorescence lifetime of porous silicon nanoparticles with various
pore diameters, particle sizes, and surface modifications.

| | Film pore size | DLS size | | |
|---|---|---|---|---|
| Sample | (nm) | (nm) | Surface | Lifetime (μs) |
| a | 7.4 | 143.4 | 2 weeks | 5.1 |
| | | | oxidized | |
| b | 9.3 | 160 | 2 weeks | 10.0 |
| | | | oxidized | |
| c | 18.1 | 159.2 | 2 weeks | 12.1 |
| | | | oxidized | |
| d | 9.3 | 58.6 | 2 weeks | 13.1 |
| | | | oxidized | |
| e | 9.3 | 130 | 3 months | 15.4 |
| | | | oxidized | |
| f | 9.3 | 167.5 | PEG | 12.0 |

The fluorescence lifetime of particles also can be effectively controlled by adsorbing or covalently attaching inorganic and organic materials that affect lifetime. wherein a fluorescence quenching or enhancing material is covalently attached to the porous Si-containing particle. Organic materials include benzene, toluene, tetrahydrofuran, xylene, diethyl ether, formic acid, benzophenone, naphthalene, pyrene, anthracene, 9,10-dimethylanthracene, ethanol, porphyrin, dodecyltrimethyl ammonium bromide, sodium dodecylsulfonate, ferrocene and derivatives. Inorganic materials include gold, silver, copper, platinum, cadmium selenide, zinc sulfide, or carbon nanotubes. Carbon nanotubes are the hardest to clear through kidneys, though, and such considerations should be considered in selecting any quenching of enhancing material.

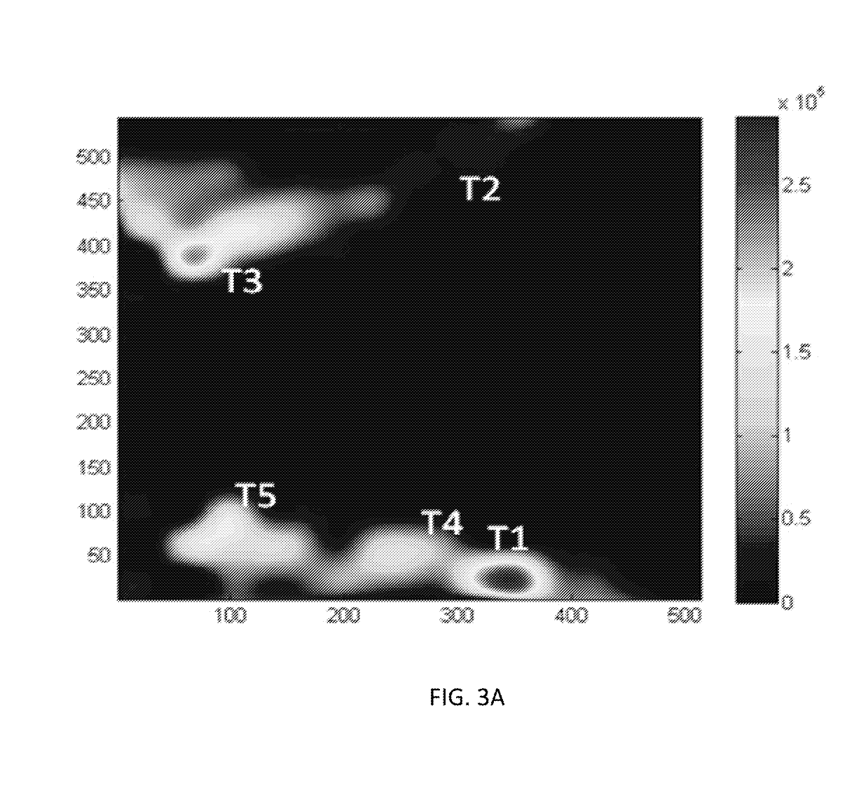

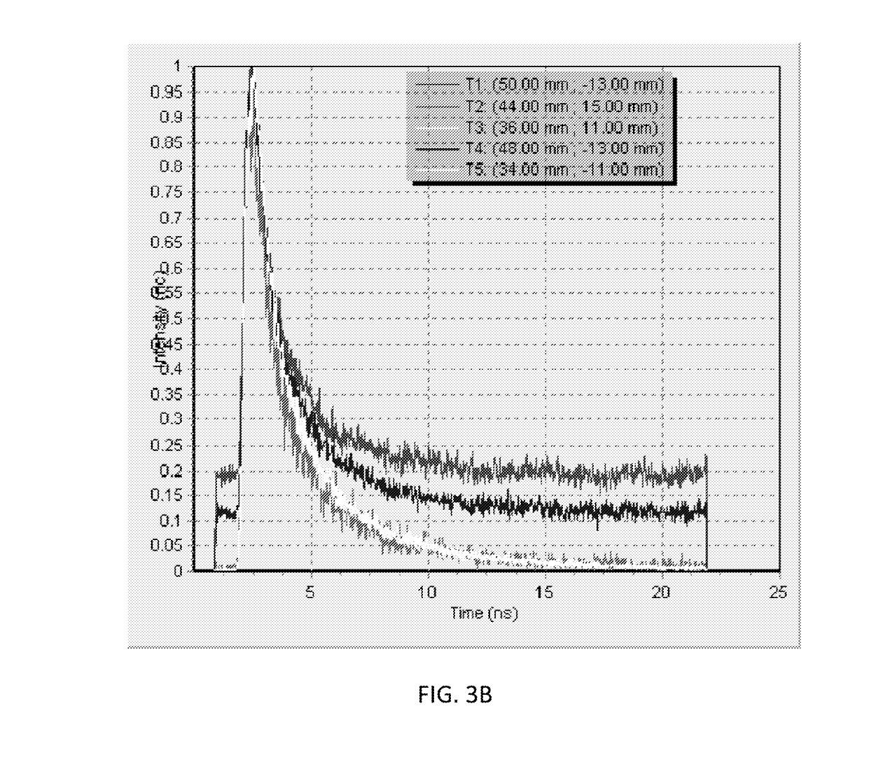

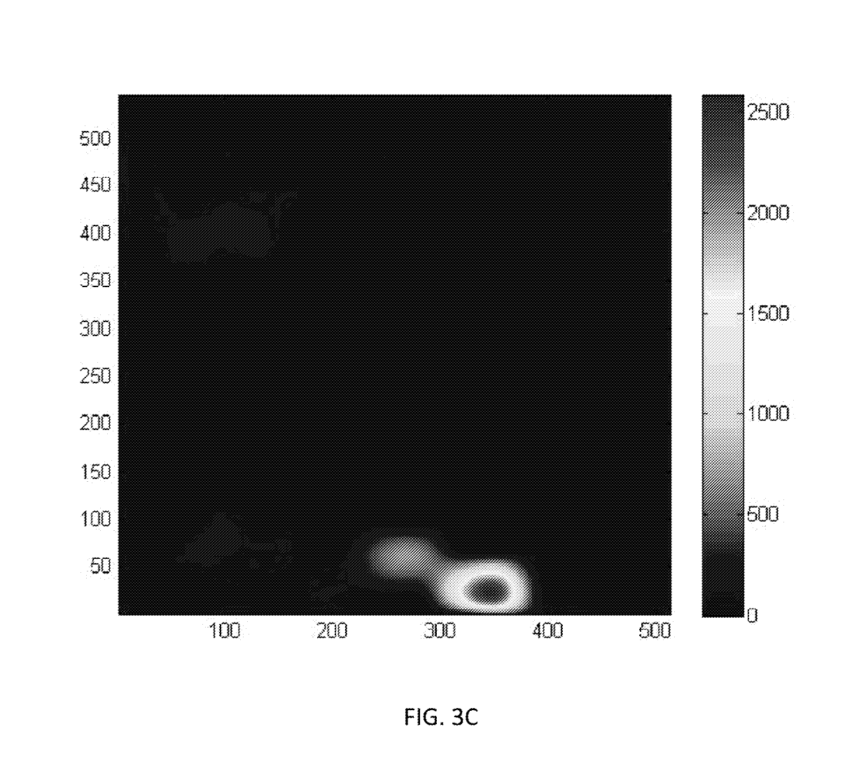

FIGS. 3A-3C show in vivo fluorescence imaging and data of porous silicon nanoparticles administered at a mouse foot pad (lower right of images at T1 and T4). FIG. 3A uses conventional wavelength based fluorescence image. The image in FIG. 3A includes strong tissue autofluorescence and excitation laser spectral bleed through (upper left and lower left at areas T2, T3 & T5) compared to the signal from porous silicon nanoparticles. FIG. 3B shows the fluorescence decay curves of the 5 different spots of the image. The porous silicon injection site (T1 and T4; upper data plots) shows little decrease of fluorescence intensity at the 15-21 ns time scale after the initial tissue autofluorescence and laser pulse peak. The autofluorescence and excitation laser spectral bleed through (remaining data plots) drops precipitously in the 5-10 ns range is negligible after about 10 μs. FIG. 3C shows a time-gated fluorescence signal after removing about the first 15 ns of the emission from FIG. 3A, showing only the long lived luminescence from porous silicon. All other fluorescence is negligible, showing that a clean signal can be provided for simple time domain analysis. No complex unmixing or filtering techniques need to be applied.

In aqueous solution borate exists in many forms. In acid and near-neutral conditions, it is boric acid, commonly written as H₃BO₃ but more correctly B(OH)₃. The pKa of boric acid is 9.14 at 25 C. Boric acid does not dissociate in aqueous solution, but is acidic due to its interaction with water molecules, forming tetrahydroxyborate.

The nanostructures can be activated to provide a luminescence. Activation of luminescence is performed in an aqueous solution. During the activation silicon oxide grows on the hydrogen-terminated porous silicon surface, generating significant luminescence attributed to quantum confinement effects and to defects localized at the Si/SiO2 interface. The preparation conditions were optimized to provide pore volume and surface area suitable for loading of therapeutics or other functional cargoes and long in vivo circulation times while maintaining an acceptable degradation rate.

Carbon grafting stabilizes porous Si against dissolution in aqueous media, but the surface should still avoid the non-specific binding of proteins and other species that can lead to opsonization or encapsulation. Carbon grafting is a preferred embodiment. Particles without the carbon treatment can be used, but are likely to have stronger non-specific binding. Reactions that place a polyethylene glycol (PEG) linker on a porous Si surface have been employed to this end. A short-chain PEG linker yields a hydrophilic surface that is capable of passing biomolecules into or out of the pores without binding them strongly. The distal end of the PEG linker can be modified to allow coupling of other species, such as drugs, cleavable linkers, fluorescent quenchers or targeting moieties, to the material.

The oxides of porous Si are easy to functionalize using conventional silanol chemistries. When small pores are present (as with p-type samples), monoalkoxydimethylsilanes (RO—Si(Me)2-d R′) can be more effective than trialkoxysilanes ((RO)2Si—R′) as surface linkers. This is because trialkoxysilanes oligomerize and clog smaller pore openings, especially when the reagent is used at higher concentrations. Whereas Si—C chemistries are robust and versatile, chemistries involving Si—O bonds represent an attractive alternative for at least two reasons. First, the timescale in which highly porous SiO2 is stable in aqueous media is consistent with many short-term drug delivery applications—typically 20 min to a few hours. Second, a porous SiO₂ sample that contains no additional stabilizing chemistries is less likely to produce toxic or antigenic side effects. If it is desired that the porous Si material be stable in vivo for long periods (for example, an extended release formulation or an in vivo biosensor), Si—C chemistries such as hydrosilylation with end capping or thermal carbonization with acetylene is useful. If a longer-lived oxide matrix is desired, silicon oxides formed at higher temperatures (>700° C.) are significantly more stable in aqueous media than those formed at lower temperatures or by ozone oxidation.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A method for imaging, the method comprising:
introducing a fluorescent Si-containing particle into tissue;
applying an excitation light pulse to excite luminescence from the fluorescent Si-containing particle;
time-gated measuring of a responsive luminescence signal to identify the particle.

2. The method of claim 1, wherein the time-gated measuring comprises sensing or analyzing the luminescence signal after a time period when autofluorescence and excitation laser spectral bleed through have negligible fluorescence.

3. The method of claim 2, wherein the period is at least about 10 nanoseconds after cessation of the excitation light pulse.

4. The method of claim 3, wherein the period is in the range of 10 nanoseconds to 1 millisecond.

5. The method of claim 1, wherein said introducing comprises introducing into tissue in vivo.

6. The method of claim 1, wherein said introducing comprises introducing a plurality of groups of fluorescent Si-containing particles with separate groups having different photoemission lifetimes.

7. The method of claim 1, wherein the fluorescent Si-containing particle comprises a biodegradable porous Si-containing nanostructure comprising a luminescence emission maximum that appears in the wavelength range of about 400 nm to about 1000 nm and a luminescence lifetime between about 10 nanosecond to 1 millisecond.

8. The method of claim 1, wherein the fluorescent Si-containing particle comprises silicon dioxide in whole or in part.

9. The method of claim 1, wherein the fluorescent Si-containing particle comprises a particle size of between about 0.001 μm and 100 μm.

10. The method of claim 1, wherein the fluorescent Si-containing particle comprises is coated or encapsulated within an organic material.

11. The method of claim 5, wherein the organic material comprises one of dextran, aminated dextran, poly(ethylene oxide), a polypeptide, DNA, RNA, polylactic acid, polyglycolic acid, collagen, fibrin, co-polymers of polylactic acid and polyglycolic acid, co-polymers of dextran and polylactic acid, chitosan, protein A, streptavidin, neutravidin, and avidin.

12. The method of claim 1, wherein a fluorescence quenching or enhancing material is covalently attached to the fluorescent Si-containing particle.

13. The method of claim 1, wherein a fluorescence quenching or enhancing material is physically absorbed in or on the fluorescent Si-containing particle.

14. The method of claim 1, wherein an organic material that affects luminescence lifetime of the fluorescent Si-containing particle is covalently attached to the fluorescent Si-containing particle.

15. The method of claim 1, wherein an organic material that affects luminescence lifetime of the fluorescent Si-containing particle is physically absorbed in or on the fluorescent Si-containing particle.

16. The method of claim 1, wherein an inorganic material that affects luminescence lifetime of the fluorescent Si-containing particle is covalently attached to the fluorescent Si-containing particle.

17. The method of claim 1, wherein an inorganic material that affects luminescence lifetime of the fluorescent Si-containing particle is physically absorbed in or on the fluorescent Si-containing particle.

18. The method of claim 1, wherein the fluorescent Si-containing particle further comprises an organic material that is one of or a derivative of one of benzene, toluene, tetrahydrofuran, xylene, diethyl ether, formic acid, benzophenone, naphthalene, pyrene, anthracene, 9,10-dimethylanthracene, ethanol, porphyrin, dodecyltrimethyl ammonium bromide, sodium dodecylsulfonate, fluorescein, ruthenium tris-bipyridine or ferrocene.

19. The method of claim 1, wherein the fluorescent Si-containing particle further comprises an inorganic material that contains one of gold, silver, copper, platinum, iron, cobalt, chromium, manganese, zinc, ruthenium, cadmium selenide, zinc sulfide, elemental carbon, or carbon nanotubes.
